(19) United States
(12) Patent Application Publication
(10) Pub. No.: US 2009/0275073 A1
(43) Pub. Date: Nov. 5, 2009

(54) DIAGNOSIS DEVICE AND DIAGNOSIS METHOD

(75) Inventor: Takahiro SHIOYAMA, Tokyo (JP)

Correspondence Address: KIMBLE INTELLECTUAL PROPERTY LAW, PLLC, 1701 PENNSYLVANIA AVE., NW, SUITE 300, WASHINGTON, DC, 20006, US

(73) Assignee: Nihon Kohden Corporation, Tokyo (JP)

(21) Appl. No.: 12/432,680

(22) Filed: Apr. 29, 2009

(30) Foreign Application Priority Data
Apr. 30, 2008 (JP) 2008-119269

(51) Int. Cl. C12Q 1/02; C12M 1/34

(52) U.S. Cl. 435/29; 435/288.7

(57) ABSTRACT

A diagnosis device includes: a pretreating portion configured to suspend tissue to obtain a suspension; and an analyzing portion configured to analyze the suspension.

### BACKGROUND OF THE INVENTION

The present invention relates to a diagnosis device and method which, when extracted tissue is loaded, can be automatically used in diagnosis such as diagnosis of cancer.

After a pathological specimen is prepared, a cytotechnologist or a pathologist performs pathology by using a tissue slice.

In the case where pathology is performed as described above, however, the cytotechnologist or the pathologist is required to have a skillful technique, and there is a problem in that the pathology is performed in a different manner depending on the difference in the skills.

In processes from tissue extraction to pathology, techniques such as tissue fixation, preparation of section, and stain are necessary, and works must be continued to be manually performed for a certain time period. By contrast, a related-art device which isolates cells from a tissue slice to obtain isolated cells is known. Such a device is used for obtaining a cell suspension by a movable dissociated element such as a blade, and configured by a single container (see JP-A-2007-505631, particularly [0025]).

Therefore, it is difficult to obtain suspension of a desired state by changing or adjusting steps such as tissue fixation, preparation of section, and stain. Furthermore, the suspension must be supplied to a flow cytometer while the container is held by a hand. Therefore, development of an automated pathology device is requested.

### SUMMARY

It is therefore an object of the invention to provide a diagnosis device and method which are configured so as to, when extracted tissue is loaded, automatically analyze suspended cells.

In order to achieve the object, according to the invention, there is provided a diagnosis device comprising:

a pretreating portion configured to suspend tissue to obtain a suspension; and

an analyzing portion configured to analyze the suspension.

The pretreating portion may include a cleansing portion configured to clean the tissue.

The pretreating portion may include a chopping portion configured to chop the tissue.

The pretreating portion may include a filtering portion which filters the suspension.

The diagnosis device may further include an enzyme treating portion configured to inject an enzyme into the suspension, thereby performing an enzyme treatment.

The diagnosis device may further include an enzyme treating portion configured to inject an enzyme into the suspension that is filtered by the filtering portion, thereby performing an enzyme treatment.

The diagnosis device may further include a centrifugal separating portion configured to apply centrifugal separation to the suspension that is filtered by the filtering portion, thereby removing supernatant.

The diagnosis device may further include a centrifugal separating portion configured to apply centrifugal separation to the suspension that is subjected to the enzyme treatment performed by the enzyme treating portion, thereby removing supernatant.

The diagnosis device may further include a centrifugal separating portion configured to apply centrifugal separation to the suspension that is filtered by the filtering portion, thereby removing supernatant.

The diagnosis device may further include a centrifugal separating portion configured to apply centrifugal separation to the suspension that is subjected to the enzyme treatment performed by the enzyme treating portion, thereby removing supernatant.

The pretreating portion may include a staining portion configured to stain the suspension.

The analyzing portion may be a flow cytometer.

In order to achieve the object, according to the invention, there is also provided a diagnosis method comprising:

suspending tissue to obtain a suspension; and

analyzing the suspension.

### BRIEF DESCRIPTION OF THE DRAWINGS

FIG. 1 is a front view of a state where a flow cytometer an embodiment of a diagnosis device of the invention is exposed.

FIG. 2 is a perspective view in which the interior of the embodiment is exposed.

FIG. 3 is a perspective view showing a pretreating portion of the embodiment, in a state where the portion is exposed.

FIG. 4 is a flowchart showing an example of the operation of the pretreating portion of the embodiment.

### DETAILED DESCRIPTION OF PREFERRED EMBODIMENTS

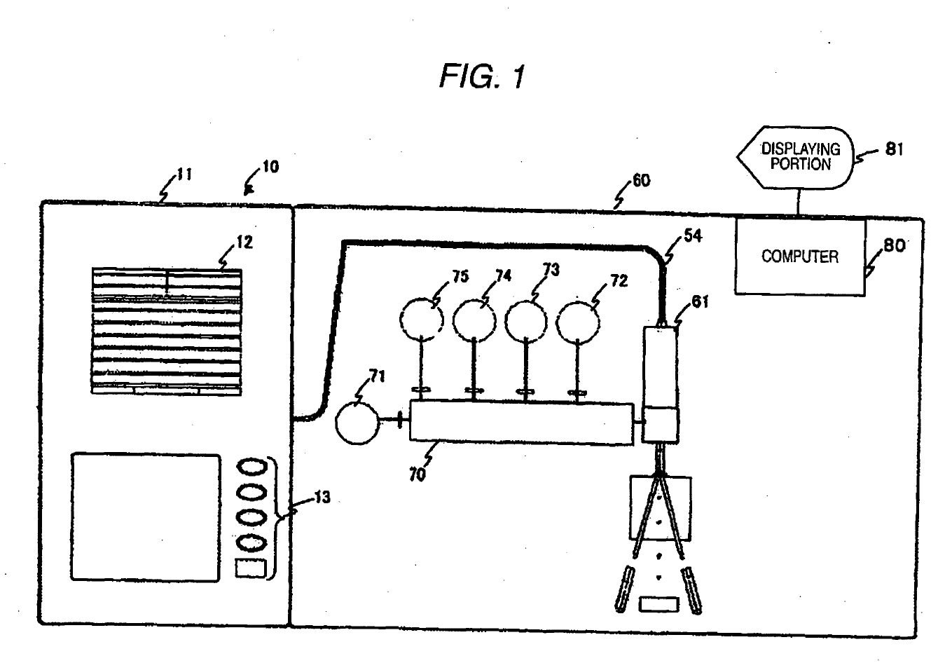

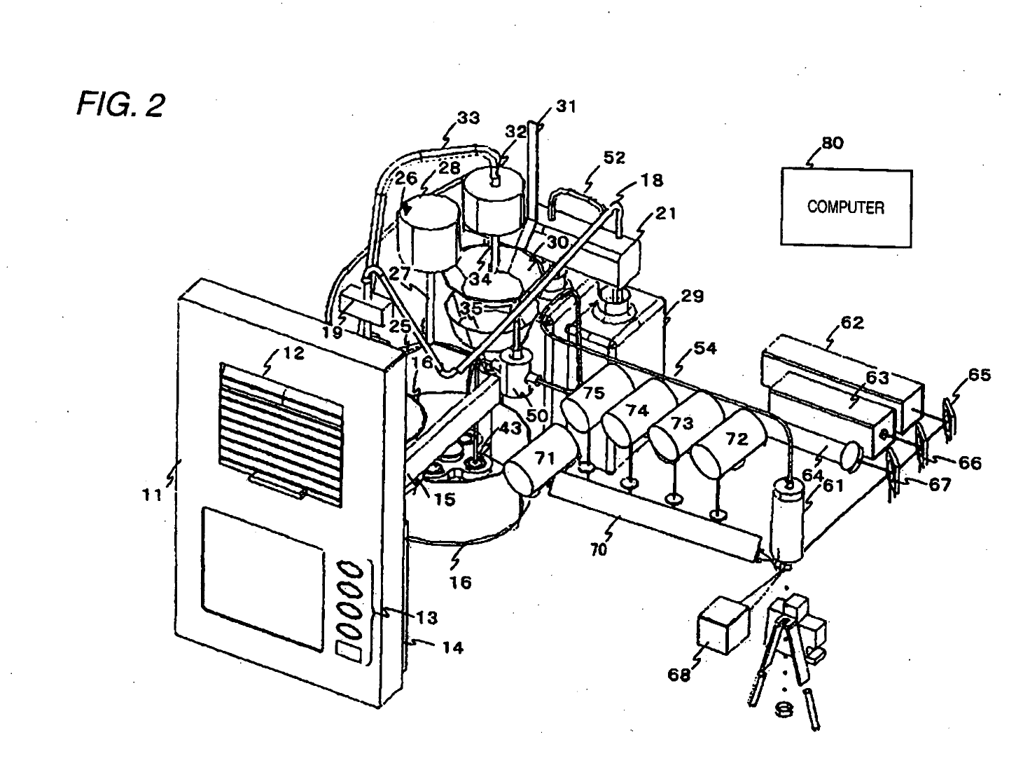

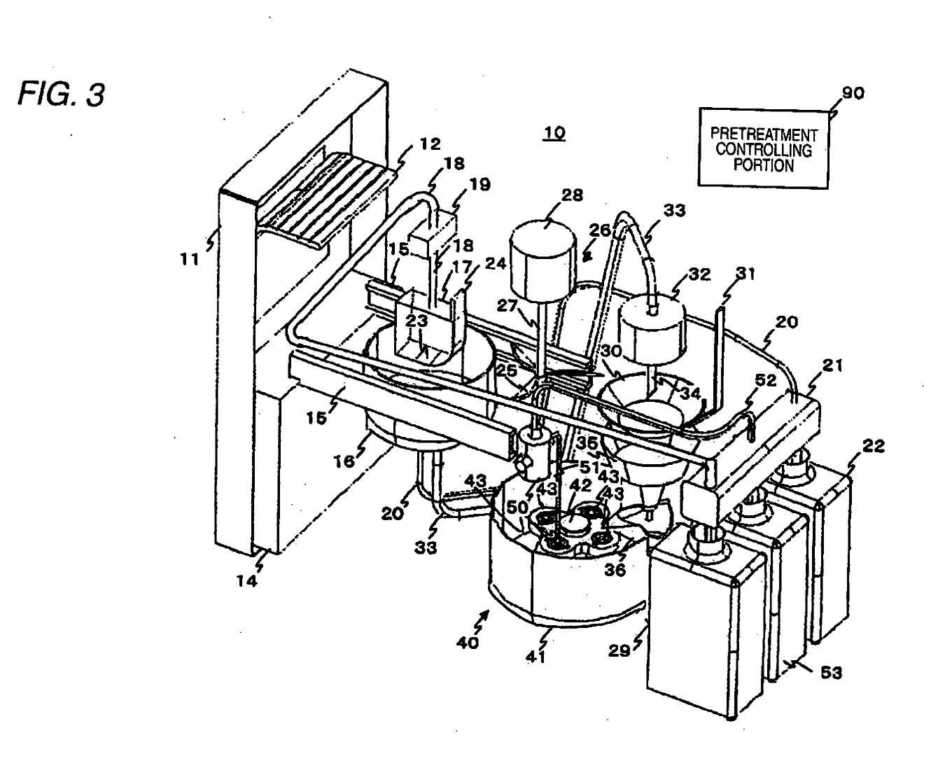

Hereinafter, an embodiment of the diagnosis device of the invention will be described with reference to the accompanying drawings. In the figures, identical components are denoted by the same reference numerals, and duplicated description is omitted. FIG. 1 is a front view of the diagnosis device, FIG. 2 is a perspective view of the diagnosis device, and FIG. 3 is a perspective view of a pretreating portion 10 which constitutes main portions of the diagnosis device. As shown in FIGS. 1 and 2, the diagnosis device includes a flow cytometer 60, in addition to the pretreating portion 10.

In a front panel 11 of the pretreating portion 10, a slide door 12 for loading extracted tissue is disposed, and operation buttons 13 through which instructions for pretreatment is to be given are provided. A base 14 is disposed in a lower portion of the rear face of the front panel 11. Two rails 15 which extend from the rear side of the front panel 11 are disposed on the base 14.

A buffer tank 16 is disposed in a position between the two rails 15 so as to be slidable along the rails 15. A driving source is disposed in at least one of the buffer tank 16 and the rails 15.

An upper portion of the buffer tank 16 is opened. Above a position (first position) where the buffer tank 16 is moved closest to the base 14, for example, a basket 17 which is formed by a stainless steel mesh is hung by a pipe 18 in a manner that the basket is made vertically movable by the pipe 18 so that the basket can be fluctuated at a position of a predetermined height in the buffer tank 16. A platform scale 19 is disposed in an upper portion of the pipe 18, and the weight of tissue which is loaded into the basket 17 can be measured. The pipe 18 is connected through a pump 21 to a reservoir tank 29 for a buffer solution such as PBS (phosphoric acid buffered saline), and the buffer solution can be supplied from the reservoir tank 29 into the buffer tank 16.

A waste solution line 20 is coupled to a bottom portion of the buffer tank 16, and communicates with a waste solution tank 22 through the pump 21. A hole 23 is formed in a bottom portion of the basket 17, and a slide cover 24 having a mechanism which makes the hole 23 openable and closable is disposed. The buffer tank 16, the basket 17, the pipe 18, the reservoir tank 29 for the buffer solution, and the like constitute a cleansing portion which cleanses the tissue with the solution.

A cutter 26 which has a blade 25 at the tip end is hung above a position (second position) where the buffer tank 16 is moved remotest on the rails 15 from the base 14. The cutter 26 includes a movement mechanism which vertical moves. The blade 25 is coupled to a motor 28 via a shaft 27 so as to be rotated. The buffer tank 16 and the cutter 26 constitute a chopping/suspending portion which receives a tissue slice from the above-described cleansing portion, and which chops the tissue slice with the rotary blade to suspend the tissue.

At a third position which is more separated from the base 14 than the second position, and which is at an approximately same level as the rails 15, a nylon mesh filter 30 is hung by an arm 31. An aspiration and discharge device 32 is disposed above the nylon mesh filter 30. An aspiration line 33 which is coupled to the bottom portion of the buffer tank 16 is connected to the aspiration and discharge device 32 so that the suspension in the buffer tank 16 can be aspirated through the aspiration line 33.

A nozzle 34 is disposed in the lower end face of the aspiration and discharge device 32. The suspension which is aspirated from the buffer tank 16 can be discharged through the nozzle 34 into the nylon mesh filter 30. An infundibulum 35 is disposed below the nylon mesh filter 30. A gutter 36 is disposed at the tip end of the infundibulum 35. The nylon mesh filter 30 and the aspiration and discharge device 32 constitute a filtering portion which performs filtration by means of the mesh. The nylon mesh filter 30 can be moved to a position outside the space between the nozzle 34 and the infundibulum 35, by a movement mechanism coupled to the arm 31.

A centrifugal separator 40 is placed on a floor face at a middle position between the third and second positions. The centrifugal separator 40 includes a rotation shaft 42 in the middle of a bottom portion of a bottomed cylindrical basket 41, and a motor which rotates the rotation shaft 42. Several solution reservoirs 43 are placed in the periphery of the rotation shaft 42. Each of the solution reservoirs 43 can be positioned to a tip end portion of the gutter 36 by the rotation to receive the supply of the suspension, and is provided with a cover which is opened and closed by an opening and closing mechanism. The centrifugal separator 40 constitutes a centrifugal separating portion which applies centrifugal separation to the suspension, thereby removing supernatant, and which further performs a suspending process.

In a space which is above the solution reservoirs 43, and which is located at a position where the suspension is not supplied from the tip end portion of the gutter 36, a staining portion 50 is disposed to inject an enzyme and a fluorescent stain solution into the suspension, thereby performing an enzyme/stain process. The staining portion 50 is configured so as to be vertically movable. An aspiration/discharge line 51 for the cell suspension is connected to the staining portion 50. The staining portion 50 is configured so that, when the portion is downward moved, the tip end of the aspiration/discharge line 51 is lowered to a position where the line can aspirate the cell suspension in the solution reservoirs 43 which is below the staining portion 50.

An enzyme/stain solution supply line 52 is connected to the staining portion 50, and communicates through the pump 21 with an enzyme/stain solution tank 53. The staining portion 50 aspirates a stain solution from the enzyme/stain solution tank 53 through the enzyme/stain solution supply line 52 and the pump 21, and mixes the stain solution with the cell suspension which is aspirated from the aspiration/discharge line 51 to perform an enzyme treatment. The cell suspension which has undergone the enzyme treatment is supplied to a flow cell 61 of the flow cytometer 60 through a cell suspension supply line 54.

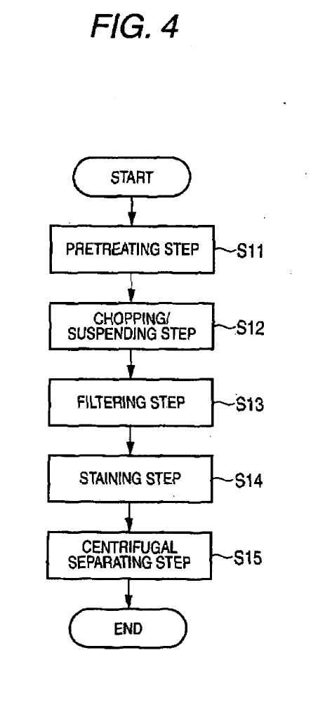

In the above-described pretreating portion 10, a pretreatment controlling portion 90 controls the various portions. The pretreatment controlling portion 90 is configured by a computer, and controls the various portions of the pretreating portion 10. In the embodiment, for example, the control is performed by a program corresponding to the flowchart shown in FIG. 4. Therefore, the operation will be described with reference to the flowchart.

First, the examiner opens the slide door 12, loads extracted tissue into the basket 17, closes the door 12, and operates a start button of the operation buttons 13. As a result, a pretreating step S11 is performed. In the pretreating step S11, the tissue is weighed by the platform scale 19, and the pretreatment controlling portion 90 receives and holds the weighing result.

In the pretreating step S11, next, the buffer solution is supplied from the reservoir tank 29 for the buffer solution into the buffer tank 16 through the pipe 18, and the basket 17 is lowered by the pipe 18 to be immersed in the buffer solution, and fluctuated so that the tissue is cleansed for a predetermined time period. Then, the basket 17 is raised by the pipe 18, and the cleansing solution (buffer solution) is recovered by the pump 21 from the waste solution line 20 to the waste solution tank 22.

In the pretreating step S11, furthermore, the buffer solution the amount of which corresponds to the weight of the tissue is supplied through the pipe 18 into the buffer tank 16, the basket 17 is lowered by the pipe 18 to be immersed in the buffer solution, and the slide cover 24 is opened, so that the tissue slice is loaded into the buffer solution of the buffer tank 16.

Next, a chopping/suspending step S12 for the tissue slice is executed. In the chopping/suspending step S12, the buffer tank 16 is moved along the rails 15 to be located at a position immediately below the blade 25 of the cutter 26. Then, the cutter 26 is lowered into the buffer solution of the buffer tank 16, and the blade 25 is rotated to chop and suspend the tissue. The pretreatment controlling portion 90 controls the speed of rotation and the time period in accordance with previously given information indicative of the kind of the tissue (for example, a plant, an animal, or a human, or a region (an internal organ, or skin)), and the weight of the tissue which is measured as described above, thereby obtaining a suspension of a desired state.

Next, the process proceeds to a filtering step S13. Before the filtering step S13, the cutter 26 is raised and moved to the home position. In the filtering step S13, the nylon mesh filter 30 is positioned between the nozzle 34 of the aspiration and discharge device 32 and the infundibulum 35, and the aspiration and discharge device 32 aspirates the solution of the buffer tank 16 through the aspiration line 33, and discharges the suspension from the nozzle 34 toward the nylon mesh filter 30 to perform filtration. The solution reservoirs 43 of the centrifugal separator 40 are sequentially positioned in the tip end portion of the gutter 36, and the filtered suspension is supplied through the gutter 36 to the solution reservoirs 43 to be stored therein.

Next, a staining step S14 is executed. In the staining step S14, the staining portion 50 is lowered, and sequentially aspirates the suspension stored in tho solution reservoirs 43 of the centrifugal separator 40 by means of the aspiration/discharge line 51 to hold them. An incubation (staining process) in which a predetermined amount of the stain solution is supplied from the enzyme/stain solution tank 53 through the enzyme/stain solution supply line 52 and the pump 21 to be mixed with the suspension is carried out. The pretreatment controlling portion 90 controls the supply amounts of the enzyme and the stain solution so as to attain an adequate dilution degree corresponding to the previously given information indicative of the kind of the tissue, the weight of the tissue which is measured in the above, etc.

Next, a centrifugal separating step S15 is performed. In the centrifugal separating step S15, the staining portion 50 discharges the cell suspension which has undergone the staining process, into the solution reservoirs 43 of the centrifugal separator 40 through the aspiration/discharge line 51, and thereafter is raised to return to the home position. The covers of the solution reservoirs 43 are closed, and centrifugal separation is performed by rotation about the rotation shaft 42. The pretreatment controlling portion 90 controls the speed of rotation and the time period in accordance with the previously given information indicative of the kind of the tissue, the weight of the tissue which is measured in the above, etc. Then, the staining portion 50 is again lowered, the staining portion 50 sequentially aspirates the cell suspension in which supernatant is removed from the suspension stored in the solution reservoirs 43 of the centrifugal separator 40, by means of the aspiration/discharge line 51, and supplies the cell suspension to the flow cell 61 of the flow cytometer 60.

The process by the flowchart of FIG. 4 is a mere example. At least one of the filtering step S13, the staining step S14, and the centrifugal separating step S15 may be omitted. When the filtering step S13 is to be omitted, the nylon mesh filter 30 may be placed at a retracted position. When the staining step S14 is to be omitted, the enzyme may not be supplied. When the centrifugal separating step S15 is to be omitted, the centrifugal separating step S15 may not be performed.

The sequence of the staining step S14 and the centrifugal separating step S15 may be changed. When a suspension discharge line extending from the staining portion 50 into the buffer tank 16 is disposed, the filtering step S13 may be repeated many times.

The progress of the steps may be set as a program in the pretreatment controlling portion 90. Alternatively, each time one of the steps is ended, the examiner may instruct the next step by using the operation buttons 13. In the alternative, the processes from the loading of tissue to the supply of the cell suspension to the flow cytometer 60 are not fully automatically performed, but, in the steps, the process contents such as the rotation number, and the process time period are automatically determined, and a cell suspension of the desired state can be obtained and supplied to the flow cytometer.

The flow cytometer 60 includes the flow cell 61, laser irradiation devices 62 to 64, dichroic mirrors 65 to 67, a forward-scattered light detection sensor 68, a mirror block 70, a side-scattered light detection sensor 71, and fluorescence detection sensors 72 to 75. In the flow cell 61, the cell suspension flows through the middle of a sheath liquid flow, and cells flow through a laser irradiation portion 69 while being arranged in one row, and then discharged from the flow cell 61 to be formed as a liquid droplet, with the result that the cells are sorted.

Laser beams emitted from the laser irradiation devices 62 to 64 reach the laser irradiation portion 69 of the flow cell 61 through the respective dichroic mirrors 65 to 67. The laser beams are scattered by the cells flowing through the laser irradiation portion 69.

The forward-scattered light detection sensor 68 is placed at a position which is on an extension of the optical path of the laser beams reaching the laser irradiation portion 69 through the dichroic mirrors 65 to 67. The mirror block 70 is placed in an optical path of side-scattered light which intersects at 90 degrees with the above-described optical path.

The mirror block 70 separates the light beams in accordance with the wavelengths to be detected by the side-scattered light detection sensor 71 and the fluorescence detection sensors 72 to 75, and guides the separated light beams to the side-scattered light detection sensor 71 and the fluorescence detection sensors 72 to 75. A cell scatters forward-scattered light of an intensity corresponding to the size of the cell. The forward-scattered light detection sensor 68 which receives the forward-scattered light detects a potential corresponding to the intensity, and sends it to a computer 80. The side-scattered light detection sensor 71 and the fluorescence detection sensors 72 to 75 introduce light of the respective wavelengths to be detected, convert the light to potentials, and send them to the computer 80.

In the computer 80, the potentials sent from the forward-scattered light detection sensor 68, the side-scattered light detection sensor 71, and the fluorescence detection sensors 72 to 75 are A/D converted to form histograms, and a dot plot in which the forward-scattered light histogram and the side-scattered light histogram are overlapped with each other is produced and displayed on a displaying portion 81. In the computer 80, furthermore, fluorescence data are produced on the basis of the outputs of the fluorescence detection sensors 72 to 75, and displayed on the displaying portion 81.

Each of the cells is classified into a normal cell and a cancer cell depending on the measurements of forward-scattered light, side-scattered light, and various fluorescences, and a cancer diagnosis of the tissue can be performed on the basis of the cells in the suspension.

Of course, the flow cytometer 60 may further have other related-art functions other than the above-described functions, and output various analysis data required in various diagnoses. As described above, according to the invention, when an extracted tissue slice is loaded, isolated and suspended cells can be automatically supplied to the flow cytometer, and a histogram and a dot plot of the cell sizes and fluorescences can be produced and output. Therefore, data necessary for various diagnoses can be obtained without requiring any skill.

According to an aspect of the invention, a diagnosis device and system can automatically analyze suspended cells when tissue is loaded therein.

What is claimed is:

1. A diagnosis device comprising:
a pretreating portion configured to suspend tissue to obtain a suspension; and
an analyzing portion configured to analyze the suspension.

2. The diagnosis device according to claim 1, wherein the pretreating portion includes a cleansing portion configured to clean the tissue.

3. The diagnosis device according to claim 1, wherein the pretreating portion includes a chopping portion configured to chop the tissue.

4. The diagnosis device according to claim 1, wherein the pretreating portion includes a filtering portion which filters the suspension.

5. The diagnosis device according to claim 3, further comprising: an enzyme treating portion configured to inject an enzyme into the suspension, thereby performing an enzyme treatment.

6. The diagnosis device according to claim 4, further comprising: an enzyme treating portion configured to inject an enzyme into the suspension that is filtered by the filtering portion, thereby performing an enzyme treatment.

7. The diagnosis device according to claims 4, further comprising: a centrifugal separating portion configured to apply centrifugal separation to the suspension that is filtered by the filtering portion, thereby removing supernatant.

8. The diagnosis device according to claims 5, further comprising: a centrifugal separating portion configured to apply centrifugal separation to the suspension that is subjected to the enzyme treatment performed by the enzyme treating portion, thereby removing supernatant.

9. The diagnosis device according to claims 6, further comprising: a centrifugal separating portion configured to apply centrifugal separation to the suspension that is filtered by the filtering portion, thereby removing supernatant.

10. The diagnosis device according to claims 6, further comprising: a centrifugal separating portion configured to apply centrifugal separation to the suspension that is subjected to the enzyme treatment performed by the enzyme treating portion, thereby removing supernatant.

11. The diagnosis device according to claim 1, wherein the pretreating portion includes a staining portion configured to stain the suspension.

12. The diagnosis device according to claim 1, wherein the analyzing portion is a flow cytometer.

13. A diagnosis method comprising:
suspending tissue to obtain a suspension; and
analyzing the suspension.
